(19) United States
(12) Patent Application Publication
(10) Pub. No.: US 2018/0289624 A1
(43) Pub. Date: Oct. 11, 2018

(54) PROCESS OF PRODUCTION OF A FORMULATION COMPRISING PHYSIOLOGICALLY ACTIVE INORGANIC METAL SALTS

(71) Applicant: DSM IP Assets B.V., Heerlen (NL)

(72) Inventors: Markus BECK, Kaiseraugst (CH); Andrea BULBARELLO, Kaiseraugst (CH); Kevin PRUDENCE, Kaiseraugst (CH); Loni SCHWEIKERT, Kaiseraugst (CH); Kai URBAN, Kaiseraugst (CH)

(21) Appl. No.: 15/756,619

(22) PCT Filed: Sep. 9, 2016
(86) PCT No.: PCT/EP2016/071322

§ 371 Date: Mar. 1, 2018

(30) Foreign Application Priority Data
Sep. 9, 2015 (EP) 15184422.2

(51) Int. Cl. A61K 9/16; A61K 33/26; A23L 33/16

(52) U.S. Cl. CPC A61K 9/1617; A61K 33/26; A23V 2002/00; A23L 33/16; A61K 9/1694

(57) ABSTRACT

This invention relates to a process for producing solid formulations. These solid formulations comprise physiologically active inorganic metal salts, which are known for being bad-tasting. These new solid formulation are neutral in taste. Furthermore the present invention relate to these solid formulations as well as to their use in the production of food, feed, nutritional supplement and personal care products.

This invention relates to a process for producing solid formulations. These solid formulations comprise physiologically active inorganic metal salts, which are known for being bad-tasting. These new solid formulation are neutral in taste. Furthermore the present invention relate to these solid formulations as well as to their use in the production of food, feed, nutritional supplement and personal care products.

Physiologically active inorganic metal salts are important and some of them even essential for a good and healthy diet (for humans as well as for animals). It is known to formulate such salt into various application forms.

By the term “physiologically active inorganic metal salts” we mean that the salts have a positive effect (on humans and/or animals) when administered orally or externally.

The problem which occurs with the solid formulation of such salts is that they can negatively interfere with other ingredients in the final food, feed, nutritional supplement or personal care applications. E.g., iron salts are known as catalysts for oxidative reactions, which can lead to the degradation of ingredients sensitive towards this kind of reactions. Furthermore, physiologically active inorganic metal salts can taste awful (especially for human beings).

One important species which falls into the group of physiologically active inorganic metal salts is iron(II) sulfate (or ferrous sulfate having the formula FeSO₄). FeSO₄ is for example used to treat iron deficiency. Iron deficiency (sideropenia or hypoferremia) is the one of the most common nutritional deficiency in the world.

Symptoms of iron deficiency include for example: fatigue, dizziness, pallor, hair loss, twitches, irritability, weakness, pica, brittle or grooved nails, impaired immune function, pagophagia and restless legs syndrome.

Due to the benefits of physiologically active inorganic metal salts (such as Iron(II) sulfate), there is a need for good working solid formulations which are neutral in taste.

Therefore, there is a need for improved solid formulation, wherein the physiologically active inorganic metal salts do not interact significantly with sensitive ingredients in food, feed, nutritional supplement or personal care application as well as when added to an end-market product does not taste awful for the consumer.

At the present time there are solid formulations for some physiologically active inorganic metal salts are known. For example from WO2005067730, wherein the FeSO₄ is coated with three layers (stearic acid/palm oil/stearic acid).

Surprisingly, we found a way to produce such solid formulations which are able to avoid the above mentioned disadvantages.

The present invention relates to a process for the production of a solid formulation wherein the solid formulation comprising at least one physiologically active inorganic metal salt and at least one lipophilic material (especially glycerol monostearate, which IUPAC name is 2,3-Dihydroxypropyl octadecanoate).

Therefore the present invention relates to a process (P) for the production of a solid formulation wherein the solid formulation comprises
(i) 20-40, weight-% (wt-%), based on the total weight of the solid formulation, of least one physiologically active inorganic metal salt, and
(ii) 60-80 wt-%, based on the total weight of the solid formulation, of at least one lipophilic material, characterized in that
(a) at least one physiologically active inorganic metal salt is suspended in the at least one liquid lipophilic material, and
(b) this suspension is atomized into a spray tower, wherein the air has such a temperature that the lipophilic material solidifies.

The new and improved solid formulation has also the additional advantages that the solid formulation is easy to produce .(conventional spray technology), the solid formulation has excellent properties to mask sour/bitter tastes, the solid formulation is a non-sticky powder, and the physiologically active inorganic metal salt is very well protected against moisture.

The physiologically active inorganic metal salt is preferably a Fe(II) salt.

Therefore, the present invention also relates to a process (P1), which is process (P), wherein the physiologically active inorganic metal salt is a Fe(II) salt.

More preferably the therapeutically active inorganic metal salt is FeSO₄.

Therefore, the present invention also relates to a process (P2), which is process (P1), wherein the physiologically active inorganic metal salt is a FeSO₄.

In a preferred embodiment, the amount of the physiologically active inorganic metal salt in the solid formulation is 20-35 wt-%, based on the total weight of the solid formulation.

Therefore the present invention also relates to a process (P3), which is process (P), (P1) or (P2), wherein the amount of the physiologically active inorganic metal salt in the solid formulation is 20-35 wt-%, based on the total weight of the solid formulation.

In a preferred embodiment, the amount of the lipophilic material in the solid formulation is 65-80 wt-%, based on the total weight of the solid formulation.

Therefore the present invention also relates to a process (P4), which is process (P), (P1), (P2) or (P3), wherein the lipophilic material in the solid formulation is 65-80 wt-%, based on the total weight of the solid formulation.

Lipophilic material in the context of the present invention can be waxes as well as fats.

Waxes in the context of the present invention are organic compounds that characteristically consist of a long alkyl chains. Natural waxes (plant, animal) are typically esters of fatty acids and long chain alcohols. Synthetic waxes are long-chain hydrocarbons lacking functional groups.

Fats, which are used for the embodiments of the present invention, consist of a wide group of compounds that are generally soluble in organic solvents and largely insoluble in water. Hydrogenated fats (or saturated fats) in the context of the present invention are generally triesters of glycerol and fatty acids. Fatty acids are chains of carbon and hydrogen atoms, with a carboxylic acid group at one end. Such fats can have natural or synthetic origin. It is possible to hydrogenate a (poly)unsaturated fat to obtain a hydrogenated (saturated) fat.

Especially suitable waxes and fats have a drop point of from 30 to 90° C., preferably 40 to 80° C. Waxes in the context of the present invention are organic compounds that characteristically consist of a long alkyl chains. Natural waxes (plant, animal) are typically esters of fatty acids and long chain alcohols. Synthetic waxes are long-chain hydrocarbons lacking functional groups.

The drop point of a material is that temperature (in ° C.) when the material begins to melt under standardized conditions. The material is heated so long until it changes the state of matter from solid to liquid. The drop point is the temperature when the first drop is released from the material. The determination of the drop point (Tropfpunkt) is carried out as described in the standard norm DIN ISO 2176.

Preferred examples of waxes and fats suitable for the present invention are glycerine monostearate, carnauba wax, candelilla wax, palmitic acid, stearic acid hydrogenated cottonseed oil and hydrogenated rapeseed oil. These compounds can be used as such or as mixtures.

Therefore the present invention also relates to a process (P5), which is process (P), (P1), (P2), (P3) or (P4), wherein the lipophilic material are waxes and fats having a drop point of from 30 to 90° C., preferably 40 to 80° C.

Therefore the present invention also relates to a process (P5′), which is process (P5), wherein the lipophilic material are waxes and fats chosen from the group consisting of glycerine monostearate, carnauba wax, candelilla wax, palmitic acid, stearic acid hydrogenated cottonseed oil and hydrogenated rapeseed oil. These compounds can be used as such or as mixtures.

Therefore the present invention also relates to a process (P5″), which is process (P5) or (P5′), wherein the lipophilic material is glycerine monostearate.

It is clear that the lipophilic material, which are not in a liquid state need to be molten before used in the process according to the present invention.

Therefore the present invention also relates to a process (P6), which is process (P), (P1), (P2), (P3), (P4), (P5), (P5′) or (P5″), wherein the lipophilic material, which is solid at room temperature is molten before used in the process.

To melt the lipophilic material usually a temperature of above 30° C. is chosen. The temperature is depending on the melting or drop point of lipophilic material. A usual and also preferred range is between 50° C. and 100° C. (more preferably 60° C.-100° C.).

Therefore the present invention also relates to a process (P6′), which is process (P6), wherein the lipophilic material, which is solid at room temperature is heated up to a temperature of above 30° C. before used in the process.

Therefore the present invention also relates to a process (P6″), which is process (P6), wherein the lipophilic material, which is solid at room temperature is heated up to a temperature of between 50° C. and 100° C. (more preferably between 60° C. and 100° C.).

Therefore the present invention also relates to a process (P6′″), which is process (P6), wherein the lipophilic material, which is solid at room temperature is heated up to a temperature of between 60° C. and 100° C.

The average particle sizes (d50) of the particles of the solid formulation obtained by any of the process (P), (P1), (P2), (P3), (P4), (P5), (P5′), (P5″), (P6), (P6′), (P6″) and/or (P6″′) is usually between 40-500 μm. The average particle sizes (d50) is measured by a MALVERN MasterSizer3000 (for all values of the present patent application).

The physiologically active inorganic metal salt is usually and preferably suspended in the liquid lipophilic material under stirring.

In step (b) of the process according to the present invention, the suspension (formed from the least one physiologically active inorganic metal salt and the least one lipophilic material) is atomized into a spray tower, wherein the air (inside the spray tower) has such a temperature that the lipophilic material solidifies.

This temperature is usually below 50° C., preferably below 40°. (Usually a range of −10° to 50° C., preferably −10° to 40° C.).

Therefore the present invention also relates to a process (P7), which is process (P), (P1), (P2), (P3), (P4), (P5), (P5′), (P5″), (P6), (P6′), (P6″) or (P6″′), wherein the suspension is atomized into a spray tower, wherein the air has s temperature of below 50° C.

Therefore the present invention also relates to a process (P7′), which is process (P), (P1), (P2), (P3), (P4), (P5), (P5′), (P5″), (P6), (P6′), (P6″) or (P6″′), wherein the suspension is atomized into a spray tower, wherein the air has a temperature of below 40° C.

Therefore the present invention also relates to a process (P7″), which is process (P), (P1), (P2), (P3), (P4), (P5), (P5′), (P5″), (P6), (P6′), (P6″) or (P6″′), wherein the suspension is atomized into a spray tower, wherein the air has a temperature of −10° to 50° C.

Therefore the present invention also relates to a process (P7″′), which is process (P), (P1), (P2), (P3), (P4), (P5), (P5′), (P5″), (P6), (P6′), (P6″) or (P6″′), wherein the suspension is atomized into a spray tower, wherein the air has a temperature of −10° to 40° C.

The solid formulation according to the present invention can also comprise other ingredients, which can be useful for the solid formulation, for the production of the solid formulation and/or the use of the solid formulation.

These other ingredients can be added at any stage to the process according to the present invention. This means they can be added to the physiologically active inorganic metal salt and/or to the lipophilic material and/or to the suspension. Optionally also some auxiliary compound can be used in the spray drying process.

Furthermore the present invention relates to a process wherein the solid formulation consists of

(i) 20-40, weight-% (wt-%), based on the total weight of the solid formulation, of least one therapeutically active inorganic metal salt, and

(ii) 60-80 wt-%, based on the total weight of the solid formulation, of glycerol monostearate.

Therefore the present invention also relates to a process (P8), which is process (P), (P1), (P2), (P3), (P4), (P5), (P5′), (P5″), (P6), (P6′), (P6″), (P6″′), (P7), (P7′), (P7″) or (P7″′), wherein the solid formulation consists of

(i) 20-40, weight-% (wt-%), based on the total weight of the solid formulation, of least one therapeutically active inorganic metal salt, and

(ii) 60-80 wt-%, based on the total weight of the solid formulation, of glycerol monostearate

A very preferred embodiment is the following process, wherein

(a) 20-40, weight-% (wt-%), based on the total weight of the solid formulation, glycerol monostearate is molted at a temperature of between 60° C. and 100° C., and

(b) 60-80 wt-%, based on the total weight of the solid formulation, FeSO₄ is suspended in the molten glycerol monostearate (by stirring), and then afterwards

(c) this suspension is atomized into a spray tower, wherein the air (in the spray tower) has a temperature of below 40° C.

Therefore the present invention also relates to a process (P9), wherein

(a) 20-40, weight-% (wt-%), based on the total weight of the solid formulation, glycerol monostearate is molted at a temperature of between 60° C. and 100° C., and

(b) 60-80 wt-%, based on the total weight of the solid formulation, FeSO₄ is suspended in the molten glycerol monostearate (by stiffing), and then afterwards

(c) this suspension is atomized into a spray tower, wherein the air (in the spray tower) has a temperature of below 40° C.

As stated above the solid formulation obtained by the process according to the present invention is in a powder form.

Furthermore the present invention also relates to the solid formulation as described above.

Therefore the present invention relates to a solid formulation (F1) comprising

(i) 20-40, weight-% (wt-%), based on the total weight of the solid formulation, of least one therapeutically active inorganic metal salt, and (

ii) 60-80 wt-%, based on the total weight of the solid formulation, of at least one lipophilc material.

Therefore, the present invention also relates to a solid formulation (F2), which is formulation (F1), wherein the physiologically active inorganic metal salt is a Fe(II) salt.

Therefore, the present invention also relates to a solid formulation (F3), which is formulation (F1), wherein the physiologically active inorganic metal salt is a FeSO₄.

Therefore the present invention also relates to a solid formulation (F4), which is formulation (F1), (F2) or (F3), wherein the amount of the physiologically active inorganic metal salt is 20-35 wt-%, based on the total weight of the solid formulation.

Therefore the present invention also relates to a solid formulation (F5), which is formulation (F1), (F2), (F3) or (F4), wherein the lipophilic material is 65-80 wt-%, based on the total weight of the solid formulation.

Therefore the present invention also relates to a solid formulation (F6), which is formulation (F1), (F2), (F3), (F4) or (F5), wherein the lipophilic material are waxes and fats having a drop point of from 30 to 90° C., preferably 40 to 80° C.

Therefore the present invention also relates to a solid formulation (F6′), which is formulation (F6), wherein the lipophilic material are waxes and fats chosen from the group consisting of glycerine monostearate, carnauba wax, candelilla wax, palmitic acid, stearic acid hydrogenated cottonseed oil and hydrogenated rapeseed oil. These compounds can be used as such or as mixtures.

Therefore the present invention also relates to a solid formulation (F6″), which is formulation (F6) or (F6′), wherein the lipophilic material is glycerine monostearate.

Therefore the present invention also relates to a solid formulation (F7), which is formulation (F1), (F2), (F3), (F4), (F5), (F6), (F6′) or (F6″), wherein the average particle sizes (d50) of the particles of the solid formulation according to the present invention is between 40-500 μm.

Therefore the present invention also relates to a solid formulation (F8) which consists of

(i) 20-40, weight-% (wt-%), based on the total weight of the solid formulation, FeSO₄, and

(ii) 60-80 wt-%, based on the total weight of the solid formulation, of glycerol monostearate.

The solid formulations (F1), (F2), (F3), (F4), (F5), (F6), (F6′), (F6″), (F7) and (F8) according to the present invention can be used as such or it can be used in any other compositions.

The solid formulations (F1), (F2), (F3), (F4), (F5), (F6), (F6′), (F6″), (F7) and (F8) as such or preferably the formulations (F1), (F2), (F3), (F4), (F5), (F6), (F6′), (F6″), (F7) and (F8) incorporated into another composition can be used as food, feed, nutritional supplement and/or personal care products.

Preferred is the use of at least one solid formulation (F1), (F2), (F3), (F4), (F5), (F6), (F6′), (F6″), (F7) and (F8) in the production of food and/or feed compositions.

The amount of the solid formulation (F1), (F2), (F3), (F4), (F5), (F6), (F6′), (F6″), (F7) and/or (F8), in the final consumer product, depends on the application and the consumer demand.

Furthermore the present invention relates to food, feed, nutritional supplement and/or personal care products comprising at least one solid formulation (F1), (F2), (F3), (F4), (F5), (F6), (F6′), (F6″), (F7) and (F8).

These products can be in any commonly known and used form.

The following examples serve to illustrate the invention.

### EXAMPLES

### Example 1

### Coated Fe(II)-Sulfate 25%

Melt 22.5 kg Glycerol Monostearate in a stirred vessel at 65-85° C.

Suspend 7.5 kg Fe(II)-sulfate into the molten Glycerol Monostearate by normal stirring until a homogeneous suspension is made (app. 20-40 min). Maintain 65-85° C. product temperature.

Feed the suspension to a spray tower. A single stage spray tower is sufficient. If needed use trace heated pipes to prevent from the suspensions from solidifying inside the pipes. Atomize the suspension to droplets of suitable size with an atomizer (preferable rotary atomizer) inside the spray tower. The spray tower has to be operated at an inlet air temperatures of app. 25° C. to solidify the atomized droplets of the suspension. The outlet air temperature should stay below 35° C.

Collect the solidified product (powder).

### Example 2

### Coated Fe(II)-Sulfate 33%

Melt 16.0 kg Glycerol Monostearate in a stirred vessel at 65-85° C.

Suspend 8.0 kg Fe(II)-sulfate into the molten Glycerol Monostearate by normal stirring until a homogeneous suspension is made (app. 20-40 min). Maintain 65-85° C. product temperature. Feed the suspension to a spray tower. A single stage spray tower is sufficient. If needed use trace heated pipes to prevent from the suspensions from solidifying inside the pipes. Atomize the suspension to droplets of suitable size with an atomizer (preferable rotary atomizer) inside the spray tower. The spray tower has to be operated at an inlet air temperatures of app. 25° C. to solidify the atomized droplets of the suspension. The outlet air temperature should stay below 35° C.

Collect the solidified product (powder).

1. Process for the production of a solid formulation comprising
(i) 20-40, weight-% (wt-%), based on the total weight of the solid formulation, of least one physiologically active inorganic metal salt, and
(ii) 60-80 wt-%, based on the total weight of the solid formulation, of at least one lipophilic material, wherein
(a) the lipophilic material is added in its liquid state and
(b) at least one physiologically active inorganic metal salt is suspended the molten lipophilic material, and
(c) the suspension is atomized into a spray tower, where the air has such a temperature that the lipophilic material solidifies.

2. The process according to claim 1, wherein the physiologically active inorganic metal salt is a Fe(II) salt.

3. The process according to claim 1, wherein the physiologically active inorganic metal salt is a FeSO₄.

4. The process according to claim 1, wherein the lipophilic material are waxes and fats having a drop point of from 30 to 90° C., preferably 40 to 80° C.

5. The process according to claim 1, wherein the lipophilic material are waxes and fats chosen from the group consisting of glycerine monostearate, carnauba wax, candelilla wax, palmitic acid, stearic acid hydrogenated cottonseed oil and hydrogenated rapeseed oil.

6. The process according to claim 1, wherein the lipophilic material is glycerol monostearate.

7. The process according to claim 1, wherein 20-35 wt-%, based on the total weight of the solid formulation. of least one physiologically active inorganic metal salt is used.

8. The process according to claim 1, wherein 65-80 wt-%, based on the total weight of the solid formulation, of at least one lipophilic material is used.

9. The process according to claim 1, wherein the average particle sizes (d50) of the particles of the solid formulation is between 40-500 μm.

10. The process according to claim 1, wherein the solid formulation consists of
(i) 20-40, weight-% (wt-%), based on the total weight of the solid formulation, of least one physiologically active inorganic metal salt, and
(ii) 60-80 wt-%, based on the total weight of the solid formulation, of glycerol monostearate.

11. A solid formulation comprising
(i) 20-40, weight-% (wt-%), based on the total weight of the solid formulation, of least one physiologically active inorganic metal salt, and
(ii) 60-80 wt-%, based on the total weight of the solid formulation, of at least one lipophilic material.

12. The solid formulation according to claim 11, wherein the physiologically active inorganic metal salt is a Fe(II) salt.

13. The solid formulation according to claim 11, wherein the physiologically active inorganic metal salt is a FeSO₄.

14. The solid formulation according to claim 11, wherein the lipophilic material is glycerol monostearate.

15. Use of at least one formulation according to claim 11 for the production of food, feed and/or personal care compositions.
